(19) United States
(12) Patent Application Publication
(10) Pub. No.: US 2023/0136413 A1
(43) Pub. Date: May 4, 2023

(54) Advanced Airway System Nasopharyngeal Injector

(71) Applicant: John Arthur Sievert, Chula Vista, CA (US)

(72) Inventor: John Arthur Sievert, Chula Vista, CA (US)

(21) Appl. No.: 17/513,903

(22) Filed: Oct. 29, 2021

(51) Int. Cl. A61M 16/04

(52) U.S. Cl. CPC A61M 16/0461; A61M 16/0402; A61M 2205/0205

(57) ABSTRACT

An Advanced Airway System Nasopharyngeal Injector is used to reduce the size of a Nasopharyngeal tube significantly. The Nasopharyngeal tube is folded in half. The Advanced Airway System Nasopharyngeal Injector will slide down the middle of the tube, locking a portion of the Nasopharyngeal tube into the track groove (104). The Nasopharyngeal tube reduction in size allows the tube to pass through the nasal passageway, decreasing discomfort and preventing debris from entering the tube. Once the Nasopharyngeal tube is administered into the passageway, it unfolds to fill the nasal passage. The Advanced Airway System Nasopharyngeal Injector uses a one-size-fits-all nasopharyngeal tube made from various materials or additive manufacturing to reduce weight and give the Advanced Airway System Nasopharyngeal Injector flexibility and durability.

### BACKGROUND

### 1. Field the Invention

This embodiment relates to a nasopharyngeal tube. Nasopharyngeal tube devices are used in the medical field and combat medics to open an airway for patients to breathe through. The Nasopharyngeal tube is made from various materials and sizes that require medical professionals to be trained on what patients get what size tube. The Nasopharyngeal tube is removed from the package and needs lubrication before insertion; once inserted, the tube can become clogged by debris, twist, and bend depending on the size and obstruction of the nasal passageway. The Nasopharyngeal tube has

### SUMMARY OF THIS EMBODIMENT

The nasopharyngeal airway device (NPA) is a tube typically made from silicone, rubber, or hollow plastics. Health care workers and combat veterans use the device to assist patients challenged to oxygenate or ventilate via mask ventilation. A Nasopharyngeal tube is administered to the patient via passing a nasopharyngeal tube through the nostril. The tube will pass through the nasal passageway and locate in the back of the mouth. The Nasopharyngeal tube comes in sizes and tube lengths ranging from 6 to 9 cm. The Advanced Airway System Nasopharyngeal Injector uses a one-size-fits-all tube that is folded in half. As it is folded in half, the rounded tube loop will slide up the Advanced Airway System Nasopharyngeal Injector track as the injector is pushed down the center of the Nasopharyngeal tube. Once the Advanced Airway System Nasopharyngeal Injector is loaded, it is lubricated and ready for insertion. Upon insertion, it follows the same path as the standard Nasopharyngeal tube through the nostril. The Advanced Airway System Nasopharyngeal Injector reduces the size of the tube, allowing for easier insertion. The ease of insertion reduces gag reflexes with patients that have a higher-than-average gag reflex. The Advanced Airway System Nasopharyngeal Injector reduces the chances of the Nasopharyngeal tube being clogged, for the tube is tightly wrapped around the injector, limiting space for debris to enter.

### BRIEF DESCRIPTION OF DRAWINGS

FIG. 1 is a left-side view schematic drawing with hidden lines removed. The schematic shows the left side of the embodiment.

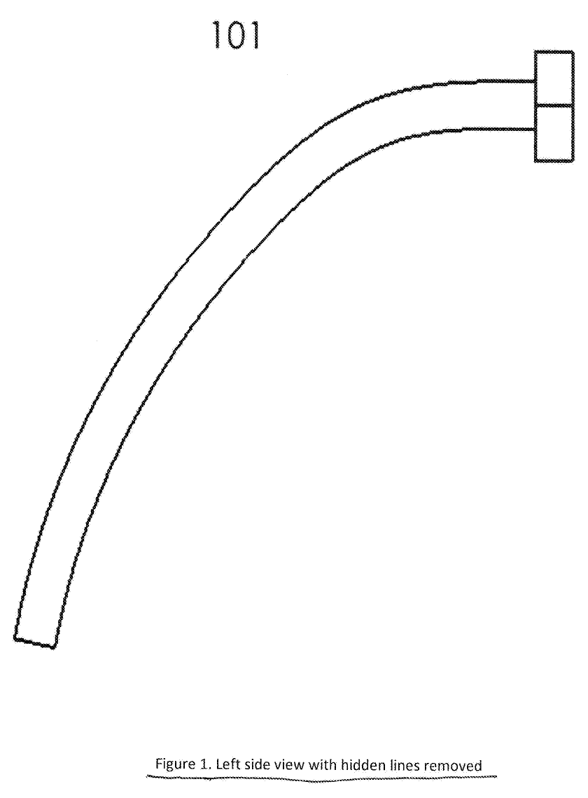

FIG. 2 is a sectional View schematic drawing A-A With Hidden Lines. The schematic shows a shaft groove (104) that travels the length of the rounded shaft (103) in the sectional view.

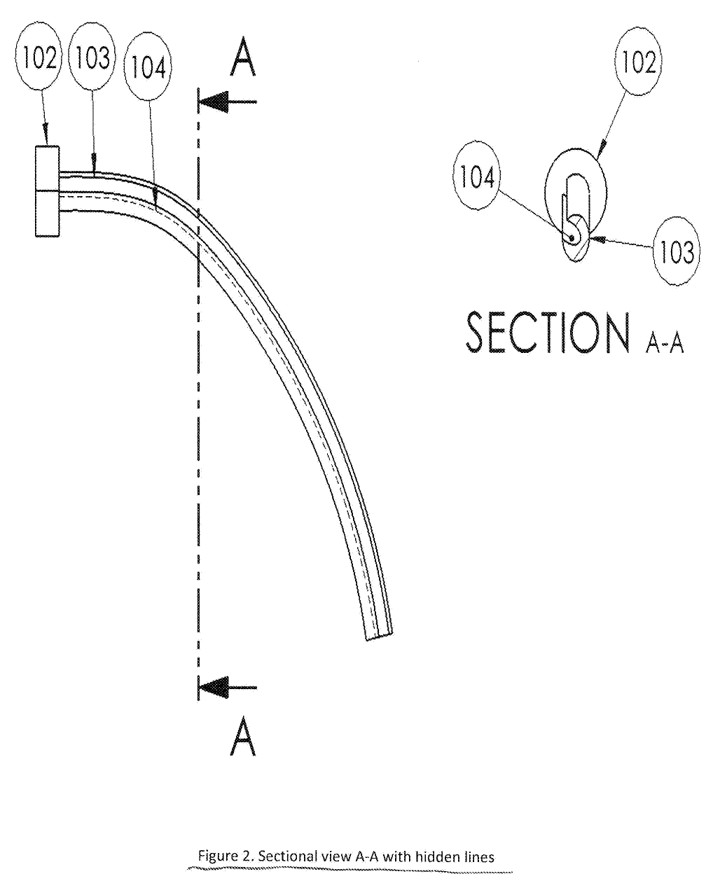

FIG. 3 is a back view schematic drawing with no hidden lines. The back view in FIG. 3 shows the shaft groove (104) that travels the length of the rounded shaft (103). The round head is shown from a back view of the embodiment.

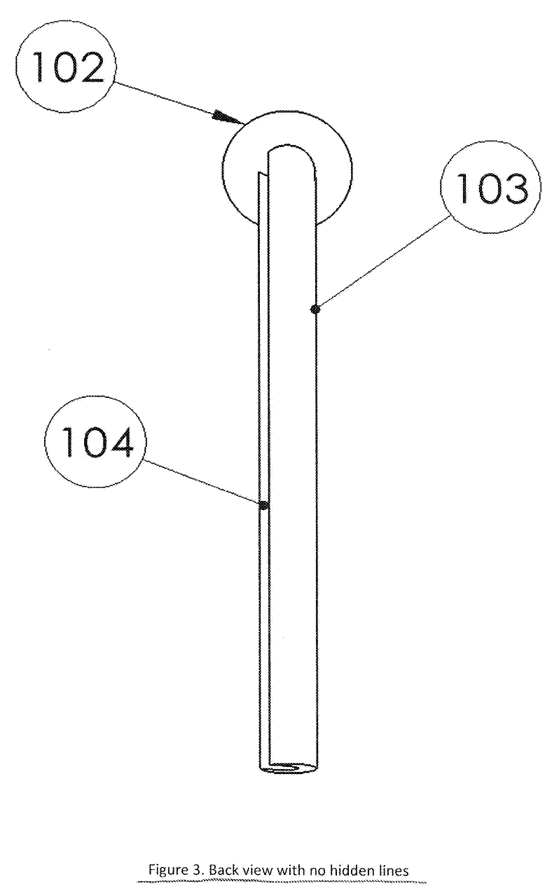

FIG. 4 is a left-side view schematic drawing with hidden lines removed. The rounded head (102) is shown from a right and back view of the embodiment.

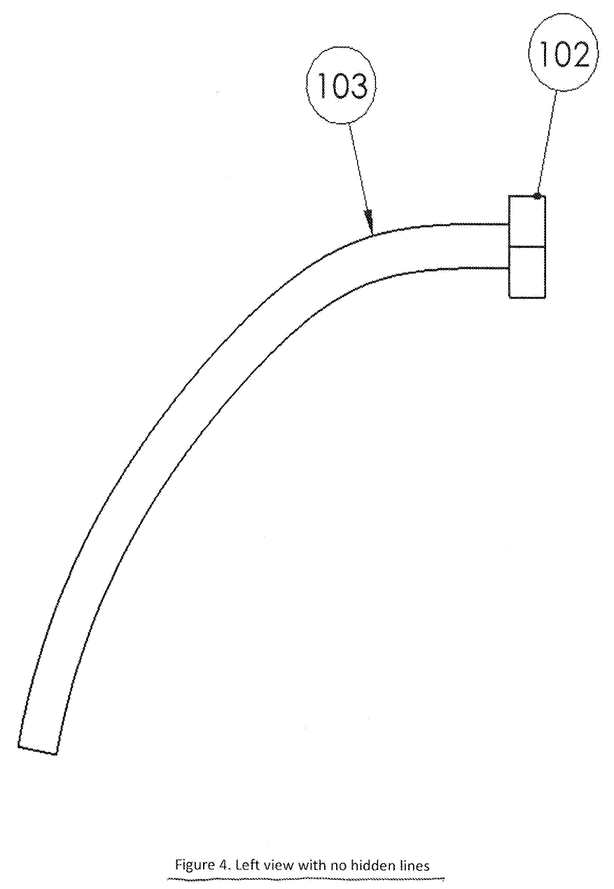

FIG. 5 is an isometric wireframe schematic drawling with no hidden lines. The schematic shows the rounded head (102) has a thickness and its roundness. The schematic shows the semi-oval shaft groove (104) in the round shaft (103) that travels the length of the rounded shaft 103).

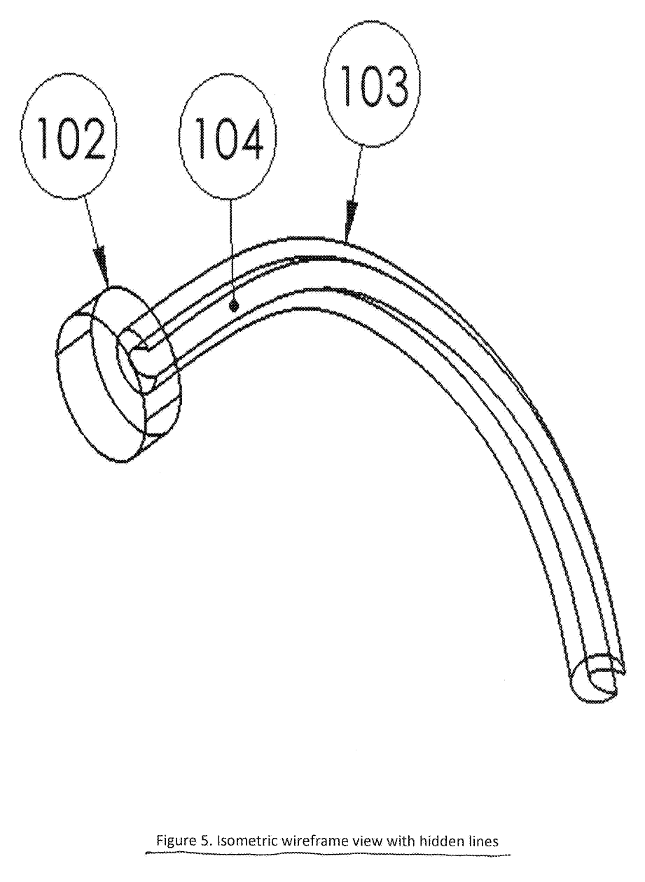

### LIST OF MAIN REFERENCE NUMERALS

101 - Advanced Airway System Nasopharyngeal Injector
102 - Rounded Guidance Head
103 - Rounded Shaft
104 - Shaft Groove

What is claimed is:

1. A injector system for a Nasopharyngeal tube wherein.
A. An injector’s rounded head is connected to a curved round shaft long enough to slide a nasopharyngeal tube up the injector.
B. The curved shaft has a groove that travels down the entire rounded shaft body where a nasopharyngeal tube is folded in on itself. The internal folded edge of the nasopharyngeal tube resides inside the shaft groove on the right side.
C. The embodiment is made from hypoallergenic material, preventing allergic reactions and anaphylaxis.
The advanced airway system nasopharyngeal is made from medical-grade material.

2. A nasopharyngeal tube delivery method reduces gaging in patients with higher than average gagging reflexes.
D. The nasopharyngeal tube size allows for easier passage through the nasal passageway. The injector eliminates the need for multiple-size nasopharyngeal tubes.
